(19) United States
(12) Patent Application Publication
(10) Pub. No.: US 2024/0268970 A1
(43) Pub. Date: Aug. 15, 2024

(54) FUSION CAGE

(71) Applicant: Zhisu Health Technology (Jiaxing) Co., Ltd., Jiaxing (CN)

(72) Inventors: Jing ZHANG, Jiaxing (CN); Kai XU, Jiaxing (CN); Lu SUN, Jiaxing (CN); Xuewei MA, Jiaxing (CN); Hao LIU, Jiaxing (CN)

(21) Appl. No.: 18/444,691

(22) Filed: Feb. 18, 2024

Related U.S. Application Data
(63) Continuation of application No. PCT/CN2022/081246, filed on Mar. 16, 2022.

(30) Foreign Application Priority Data
Sep. 13, 2021 (CN) 202111067254.1

(51) Int. Cl. A61F 2/44; A61F 2/30

(52) U.S. Cl. CPC A61F 2/4465; A61F 2002/30266; A61F 2002/30784

(57) ABSTRACT

The present invention discloses a fusion cage. The fusion cage includes a first end surface and a second end surface. The first end surface is provided with a first lattice, and at least one region of the second end surface is provided with a second lattice. The first lattice provides a pathway for attaching and supporting human tissue, facilitating inward bone ingrowth for the fusion cage. The structure of the second lattice is designed to decrease the overall elastic modulus of the product and provide a region for bone ingrowth. Different lattice layouts, with the first lattice facilitating inward bone ingrowth and the second lattice reducing bone elastic modulus, address the issue in existing fusion devices of not meeting both bone elastic modulus matching and human tissue growth simultaneously.

### TECHNICAL FILED

The present invention relates to the field of medical devices, particularly to a fusion cage.

### BACKGROUND

Currently, the lattice structure on fusion cages is designed to reduce the elastic modulus of the fusion cage and provide channels for bone ingrowth into the interior. After implantation into the human body, this facilitates better integration of the fusion cage with the human body. However, existing intervertebral fusion cages often employ a fully solid structure or lattice structures distributed on the lateral sides, resulting in a higher elastic modulus and a propensity for stress shielding. Additionally, the effectiveness of bone ingrowth is suboptimal. Therefore, the technical problem that professionals in this field need to address is how to develop a fusion cage that simultaneously meets the matching elastic modulus of bones and promotes the growth of human tissue. There is an urgent need for a fusion cage that can address the technical issues in prior art, where fusion cages are unable to simultaneously meet the matching elastic modulus of bones and promote the growth of human tissue.

### SUMMARY

The present application provides a fusion cage, aiming to address the technical issues in prior art, where fusion cages are unable to simultaneously meet the matching elastic modulus of bones and promote the growth of human tissue, by configuring lattice structures with different specifications on different end surfaces.

In one embodiment, the fusion cage has a first end surface and a second end surface, with the first end surface having a first lattice, and at least one region on the second end surface provided with a second lattice.

In one embodiment, the first end of the fusion cage has an instrument groove that opens towards one side facing an external environment. The instrument groove has a gripping portion to allow external instruments to grasp the fusion cage.

In one embodiment, the gripping portion is a cylinder, with two ends of the cylinder being connected to an inner wall of the instrument groove.

In one embodiment, the second end of the fusion cage is a tapered end, and the tip of the tapered end protrudes outward.

In one embodiment, the first end surface has at least one inclined surface reinforcement rib. The two ends of the inclined surface reinforcement rib span the first lattice and connect to the second end surface. The first end surface is either an arc-shaped surface or a flat surface with a curvature.

In one embodiment, the inclined surface reinforcement rib has protrusions facing outward from the fusion cage. The protrusions have an inclined surface on the side facing the tapered end, arranged from low to high from the second end to the first end.

In one embodiment, the at least one inclined surface reinforcement rib includes multiple inclined surface reinforcement ribs arranged at intervals between the first end and the second end.

In one embodiment, the fusion cage is a rectangular prism, with one side of the rectangular prism being the first end surface, one end of the rectangular prism being the first end, and the tapered end smoothly joining the first end surface.

In one embodiment, the first end and the second end of the fusion cage is bent in the same direction, causing the second end surface to curve into a curved surface.

In one embodiment, the first end surface has a bone graft chamber, which is a through-hole that penetrates the fusion cage.

### BRIEF DESCRIPTION OF THE DRAWINGS

FIG. 1 is a schematic diagram of the external structure of the fusion cage in one embodiment of the present invention.

FIG. 2 is a schematic diagram of the top view of the fusion cage in one embodiment of the present invention.

FIG. 3 is a schematic diagram of the first end of the fusion cage in one embodiment of the present invention.

FIG. 4 is a schematic diagram of the side view of the fusion cage in one embodiment of the present invention.

### CHARACTER REFERENCES IN THE DRAWINGS

First end surface 1
First lattice 11
   Inclined surface reinforcement rib 12
      Protrusion 121
      Inclined surface 122
   Bone graft chamber 13
Second end surface 2
   Second lattice 21
First end 3
   Instrument groove 31
   Gripping portion 32
Second end 4

### DETAILED DESCRIPTION OF THE EMBODIMENTS

In the prior art, the inventors found that the elastic modulus of current intervertebral fusion cages tends to be high, leading to stress shielding and suboptimal bone ingrowth. Addressing the technical challenge of simultaneously meeting the matching elastic modulus of bones and promoting the growth of human tissue is crucial.

The following, in conjunction with the drawings in the embodiments of this application, provides a clear and comprehensive description of the technical solution in the embodiments of this application. It is evident that the described embodiments are only a part of the embodiments of this application, not the entire embodiments. Based on the embodiments in this application, all other embodiments obtained by those skilled in the art without creative labor are within the scope of protection of this application.

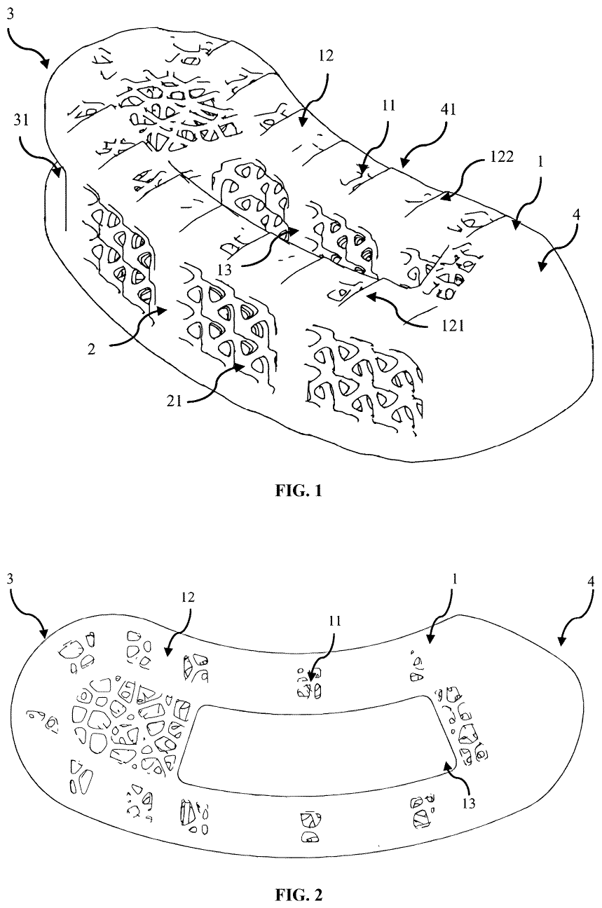

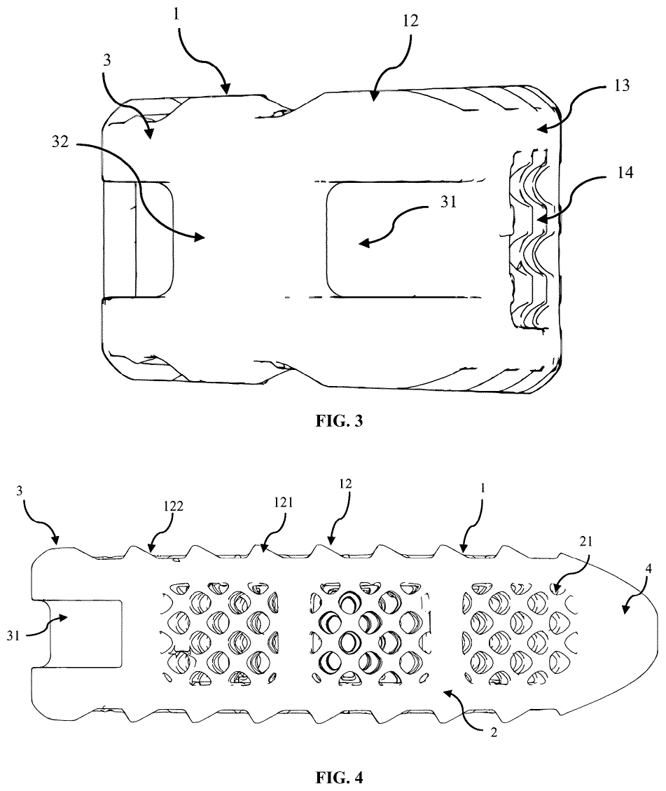

FIG. 1 is a schematic diagram of the external structure of the fusion cage in one embodiment of the present invention, FIG. 2 is a schematic diagram of the top view of the fusion cage in one embodiment of the present invention, and FIG. 3 is a schematic diagram of the first end of the fusion cage in one embodiment. As shown in FIGS. 1 and 2, and FIG. 3, in one embodiment, the present application provides a fusion cage. The fusion cage has a first end surface 1 and a second end surface 2, with the first end surface 1 having a first lattice 11 and the second end surface 2 having at least one region provided with a second lattice 21.

In this embodiment, a specific structure is disclosed for arranging the first lattice 11 on the first end surface 1. The first lattice 11 can provide a pathway for bone ingrowth between the upper and lower vertebrae. The first lattice 11 is a spatial structure constructed by struts, with gaps and other structures between the struts. The first lattice 11 can also be an essential component constituting the elastic modulus of the fusion cage. However, as mentioned earlier, the calculation of the elastic modulus is complex. Therefore, the first lattice 11 is considered a random lattice here. While the first lattice 11 reduces the overall elastic modulus of the fusion cage, it does not specify the exact numerical value of the elastic modulus. Thus, the first lattice 11 is understood to be a random lattice, as long as it provides an elastic modulus within a certain range. The calibration of the bone elastic modulus of the fusion cage comes from the second lattice 21 on the second end surface 2. Since the first lattice 11 has already reduced the bone elastic modulus of the fusion cage, the second lattice 21 compensates for the deficiency in fitting the bone elastic modulus, enabling the fusion cage to fully match the required bone elastic modulus. This approach, balancing production costs and meeting bone elastic modulus requirements through the layout of different lattices, helps address the technical problem in current technology where fusion cages struggle to balance costs and provide the required bone elastic modulus.

In one embodiment, the first end 3 of the fusion cage is provided with an instrument groove 31. The instrument groove 31 opens towards one side facing the external environment, and the instrument groove 31 is provided with a gripping portion 32 to allow external instruments to grasp and hold the fusion cage.

This embodiment provides a specific structure with an instrument groove 31 for the fusion cage. In prior art, a tool hole is usually provided at an appropriate position on the fusion cage. This tool hole is a threaded hole, and the external instrument has a corresponding bolt. After connecting the bolt to the threaded hole, the fusion cage and the external instrument are engaged together. In clinical procedures, the fusion cage is first placed between two vertebrae using the tapered end. The surgeon then adjusts the relative position of the fusion cage between the vertebrae by manipulating the external instrument. However, the rigid connection in the prior art limits the operational space for the surgeon to adjust the fusion cage. Once connected rigidly, the relative position between the fusion cage and the external instrument remains unchanged.

In this embodiment, a specific structure of the fusion cage is provided. Firstly, an instrument groove 31 is provided on the first end 3 of the fusion cage. The outer profile of the fusion cage features rounded transitions to prevent collision and damage to human tissues. The instrument groove 31 is designed as a concave structure, avoiding dead angles or protrusions that may cause impact with human tissues. Additionally, the instrument groove 31 opens towards one side, creating an opening to provide more operational space for external instruments. A gripping portion 32 is positioned within the instrument groove 31, allowing the external instrument to grip and hold the gripping portion. In this embodiment, the threaded hole connection method in the prior art is abandoned in favor of a gripping method to connect the gripping portion 32 and the external instrument. The open design of the instrument groove 31 allows for a larger connection space between the external instrument and the gripping portion 32. During clinical surgery, the surgeon places the fusion cage at the lesion site, releases the connection between the external instrument and the gripping portion 32, rotates the external instrument, grips the gripping portion 32, and repeats this process until the fusion cage is correctly positioned. By adjusting the relative position between the fusion cage and the external instrument, the surgery is facilitated, and work efficiency is improved. This addresses the technical issue in surgical procedures where the rigid connection in prior art results in time-consuming and labor-intensive adjustments when using external medical tools to adjust the orientation of the fusion cage.

In one embodiment, the gripping portion 32 is a cylinder, with two ends of the cylinder being connected to an inner wall of the instrument groove 31.

This embodiment provides a specific structure for the gripping portion 32. Using a cylindrical gripping portion 32 allows the external instrument to rotate along the axis of the cylinder. In practical applications, the space at the lesion site may be limited. Therefore, when the external instrument completely detaches from the gripping portion 32 to adjust the angle and then grips the gripping portion 32 again, although it can be achieved on the operational level, a better solution is to release the external instrument without completely detaching it from the gripping portion 32. This way, the external instrument can rotate along the axis of the cylinder, only adjusting the angle without completely disconnecting from the fusion cage. This method not only facilitates the adjustment of the relative position between the fusion cage and the external instrument, making the surgery more convenient and efficient but also avoids completely detaching the fusion cage and the external instrument, increasing the difficulty of the surgery.

In one embodiment, the second end 4 of the fusion cage is a tapered end, and the tip of the tapered end protrudes outward.

This embodiment further discloses the specific structure of the fusion cage. The tapered end is also smoothly transitioned with rounded corners, forming a smooth surface. The purpose is to reduce the difficulty of the implantation process. In clinical practice, the fusion cage is initially placed between two vertebrae using the tapered end.

FIG. 4 is a schematic diagram of the side view of the fusion cage in one embodiment of the present invention. In one embodiment, the first end surface 1 has at least one inclined surface reinforcement rib 12. The two ends of the inclined surface reinforcement rib 12 span the first lattice 11 and connect to the second end surface 2, and the first end surface 1 is either an arc-shaped surface or a flat surface with a curvature.

This embodiment discloses a specific structure with inclined surface reinforcement ribs 12 arranged on the first end surface 1. One of the functions of the inclined surface reinforcement ribs 12 is to supplement the strength of the first end surface 1. Additionally, since the first end surface 1 directly contacts human tissue after the fusion cage is implanted, it needs inclined surface reinforcement ribs 12 as protrusions to provide some resistance and friction. This helps stabilize the fusion cage during the early stages of implantation.

In one embodiment, the inclined surface reinforcement ribs 12 have protrusions 121. The protrusions 121 face outward from the fusion cage, and the protrusions 121 on the side facing the tapered end have inclined surfaces 122. The inclined surfaces 122 are arranged from the second end 4 to the first end 3, gradually increasing in height.

This embodiment provides a specific structure for the inclined surface reinforcement ribs 12. When the first end surface 1 contacts human tissue during surgery, the protrusions 121 act to resist friction. As the protrusions 121 are pushed in, the inclined surfaces 122 assist the fusion cage in further entering the space between two vertebrae. Therefore, the inclined surfaces 122 facing the side of the second end 4 are lower, and the inclined surfaces 122 facing the side of the first end 3 are higher.

In one embodiment, at least one inclined surface reinforcement rib 12 includes multiple inclined surface reinforcement ribs 12, with the multiple inclined surface reinforcement ribs 12 spaced between the first end 3 and the second end 4.

In this embodiment, a specific structure is provided with multiple inclined surface reinforcement ribs 12. Multiple inclined surface reinforcement ribs 12 are preferably arranged at equal intervals on the first end surface 4. It should be noted that the first lattice 11 is arranged in a strip between the first end 3 and the second end 4.

In one embodiment, the fusion cage is a rectangular prism, with one side being the first end surface 1, one end being the tapered end, and the tapered end smoothly connecting to the first end surface 1.

This embodiment further discloses the specific structure of the fusion cage. The rectangular prism may also have the first lattice 11 on the side relative to the first end surface 1. Additionally, to facilitate the insertion of the fusion cage between two vertebrae, the tapered end smoothly transitions to the first end surface 1, preferably, the tapered end smoothly transitions to all four sides through curved surfaces, and the tapered end can be understood as a bullet head shape.

Furthermore, in this embodiment, a specific structure is provided for setting the second lattice 21 on the second end surface 1. As mentioned earlier, the first lattice 11 is obtained in a random manner, providing a certain elastic modulus to the fusion cage. Since the first lattice 11 is arranged on the first end surface 1, which directly contacts human tissue, and is supported by inclined surface reinforcement ribs 12, the first lattice 11 also provides a certain structural strength. One of the functions of the second lattice 21 is to induce bone ingrowth, and another function is to calibrate the bone elastic modulus. The ideal state for the fusion cage is to match the different bone elastic moduli of different individuals. Therefore, the second lattice 21 will calculate the patient's bone elastic modulus through the device, and then spatially model the second lattice 21, finding structural parameters that match the overall elastic modulus of the fusion cage with the patient's bone elastic modulus. It should be noted that both the first lattice 11 and the second lattice 21 can be constructed as spatial lattice structures using struts. The gaps between the struts can be measured by pore size and can be constructed to achieve the desired elastic modulus of the first lattice 11 and the second lattice 21. Additionally, the first lattice 11 may have multiple regions spaced between the first end 3 and the second end 4, and these multiple first lattices 11 can be arranged from the first end 3 to the second end 4. The first lattice 11 also has the function of promoting the growth of human tissue. During clinical procedures, surgeons will first remove the human tissue between the upper and lower two vertebrae. Then, the fusion cage is placed between the two vertebrae, with the first end surface 4 and the second end surface 13 respectively contacting the two vertebrae, promoting bone ingrowth on both the first end surface 1 and the second end surface 2. Additionally, the instrument groove 31 is arranged between the first end surface 3 and one side surface opposite the first end surface 1. The instrument groove 31 is at a certain distance from both the first end surface 1 and that side surface. Preferably, the instrument groove 31 is arranged in the middle portion between the first end surface 1 and that side surface.

In this embodiment, the first end 3 and the second end 4 of the fusion cage are bent in the same direction to make the middle part of the fusion cage being a curved surface. When the fusion cage is placed between adjacent vertebrae, the curved surface of the middle part can be substantially consistent with the edge of the vertebrae to avoid interference with external tissues.

In one embodiment, a bone graft chamber 13 is arranged on the first end surface 1, and the bone graft chamber 13 is a through-hole that penetrates the fusion cage.

This embodiment provides a specific structure of the fusion cage with a bone graft chamber 13. The bone graft chamber 13 passes through from the first end surface 1 to the side surface on the opposite side. Meanwhile, the fusion cage may not be provided with a bone graft chamber 13, and the first lattice 11 and the second lattice 21 are extended to the center area of the fusion cage, and the inclined surface reinforcement ribs 12 pass through both ends of the fusion cage.

Those skilled in the art can understand that the various embodiments and/or features recorded in the claims can be combined and/or combined in various ways, even if such combinations or combinations are not explicitly recorded in the present application. In particular, within the spirit and teaching of the present application, various combinations and/or combinations of features recorded in various embodiments and/or claims of the present application can be made, and all such combinations and/or combinations fall within the scope disclosed in the present application.

Specific embodiments have been used to illustrate the principles and embodiments of the present invention, and the description of the above embodiments is only intended to help understand the method and core ideas of the present invention and is not intended to limit this application. For those skilled in the art, various changes, modifications, improvements, etc. can be made to the specific embodiments and applications within the spirit and principles of the present invention, and all such modifications, changes, improvements, etc. should be included in the protection scope of this application.

What is claimed is:

1. A fusion cage, characterized in that the fusion cage includes a first end surface (1) and a second end surface (2), with the first end surface (1) having a first lattice (11), and at least one region on the second end surface (2) provided with a second lattice (21).

2. The fusion cage according to claim 1, characterized in that the first end (3) of the fusion cage is provided with an instrument groove (31) that opens towards one side facing an external environment; the instrument groove (31) has a gripping portion (32) to allow external instruments to grasp the fusion cage.

3. The fusion cage according to claim 2, characterized in that the gripping portion (32) is a cylinder, with two ends of the cylinder being connected to an inner wall of the instrument groove (31).

4. The fusion cage according to claim 3, characterized in that, the second end (4) of the fusion cage is a tapered end, and the tip of the tapered end protrudes outward.

5. The fusion cage according to claim 4, characterized in that the first end surface (1) has at least one inclined surface reinforcement rib (12); two ends of the inclined surface reinforcement rib (12) span the first lattice (11) and connect to the second end surface (2); the first end surface (1) is either an arc-shaped surface or a flat surface with a curvature.

6. The fusion cage according to claim 5, characterized in that the inclined surface reinforcement rib (12) has protrusions (121) facing outward from the fusion cage; the protrusions (121) have an inclined surface (122) on the side facing the tapered end, arranged from low to high from the second end (4) to the first end (3).

7. The fusion cage according to claim 6, characterized in that the at least one inclined surface reinforcement rib (12) includes multiple inclined surface reinforcement ribs arranged at intervals between the first end (3) and the second end (4).

8. The fusion cage according to claim 7, characterized in that the fusion cage is a rectangular prism, with one side of the rectangular prism being the first end surface (1), one end of the rectangular prism being the first end (3), and the tapered end smoothly joining the first end surface (1).

9. The fusion cage according to claim 8, characterized in that the first end (3) and the second end (4) of the fusion cage is bent in the same direction, causing the second end surface (4) to curve into a curved surface.

10. The fusion cage according to claim 9, characterized in that the first end surface (1) has a bone graft chamber (13), which is a through-hole that penetrates the fusion cage.
